(19) United States
(12) Patent Application Publication
(10) Pub. No.: US 2009/0005297 A1
(43) Pub. Date: Jan. 1, 2009

(54) Aprotinin Variants

(76) Inventors: Kevin Lumb, Guilford, CT (US); Stanley Horton, Madison, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 11/659,434

(22) PCT Filed: Jul. 13, 2005
(86) PCT No.: PCT/US05/24951

§ 371 Date: Feb. 2, 2007

Related U.S. Application Data
(60) Provisional application No. 60/587,655, filed on Jul. 13, 2004.

(51) Int. Cl. A61K 38/16; C07K 14/00; C07H 21/00; C12P 21/00; A61P 7/08; C12N 1/21; C07K 16/00; C12N 15/64

(52) U.S. Cl. 514/12; 530/324; 536/23.1; 435/69.1; 435/252.33; 530/387.1; 435/320.1

(57) ABSTRACT

The present invention relates to the field of proteins that inhibit serine protease activity. The invention also relates to the field of nucleic acid constructs, vectors and host cells for producing serine protease inhibiting proteins, pharmaceutical compositions containing such proteins, and methods for their use.

This application claims benefit of U.S. Provisional Application Ser. No. 60/587,655; filed on Jul. 13, 2004, the contents of which are incorporated herein by reference in their entirety.

### FIELD OF THE INVENTION

The present invention relates to the field of proteins that inhibit serine protease activity. The invention also relates to the field of nucleic acid constructs, vectors and host cells for producing serine protease inhibiting proteins, pharmaceutical compositions containing such proteins, and methods for their use.

### BACKGROUND OF THE RELATED ART

Blood loss is a serious complication of major surgeries such as open-heart surgery and other complicated procedures. Cardiac surgery patients account for a significant proportion of transfused donor blood, and yet blood transfusion carries risks of disease transmission and adverse reactions. In addition, donor blood is expensive and demand often exceeds supply.

Aprotinin (Trasylol®) is utilized for reducing perioperative blood loss (Dietrich, et al., Thorac. Cardiovasc. Surg. 37:92-98, 1989). Aprotinin, a bovine serine protease inhibitor of the Kunitz family, is generally thought to reduce in vivo blood loss through inhibition of proteases such as plasmin. However, adverse effects, including hypotension and flushing (Bohrer, et al., Anesthesia 45:853-854, 1990) and allergic reactions (Dietrich, et al., 1989) have been reported. Furthermore, repeated use of aprotinin in patients with known immunoglobulins is not recommended (Dietrich, et al., 1989).

Aprotinin is used to reduce blood loss during cardiovascular surgeries (e.g., coronary artery bypass, off-pump, valve, vascular, lung-volume reduction and Cox-Maze procedures), orthopedic surgeries (e.g., spine, hip replacement and repair, knee replacement and tumor resection), neurosurgery, and major reconstructive (plastic) surgery.

Aprotinin is also used in the treatment of trauma (including multi-organ dysfunction and brain injury), ischemia reperfusion injury (e.g., stroke, intracerebral hemorrhage, myocardial Infarction, transplant preservation, and anterior cruciate ligament), cancer (e.g., metastasis and primary tumor suppression), lung ciliary functions (e.g., asthma, cystic fibrosis, chronic obstructive pulmonary disease and antitrypsin deficiency), and organ transplant procedures (e.g., post-cadaveric organ preservation and transplant surgery). Aprotinin is also used in applications such as fibrin glues (e.g., for use during spinal taps, treating surgical wounds, and dental surgery).

Because aprotinin is of bovine origin, there is a finite risk of inducing anaphylaxis in human patients upon re-exposure to the drug. Aprotinin is also nephrotoxic in rodents and dogs when administered repeatedly at high dose (Glasser, et al., “Verhandlungen der Deutschen Gesellschaft fur Innere Medizin, 78. Kongress,” Bergmann, Munchen, pp. 1612-1614, 1972). One hypothesis ascribes this effect to the accumulation of aprotinin due to its high net positive charge in the negatively charged proximal tubules of the kidney (WO 93/14120).

In a number of cases, it has been shown that PEGylation may reduce the immunogenicity of proteins. However, PEGylation often reduces the functional activity of the modified protein, which in the case of an antagonist such as aprotinin is undesirable. The current state of the art for PEGylated aprotinin is the nonspecific PEGylation at amine groups with one or two 5 kDa PEG modifications of a variant aprotinin (T11 D, K15R, R17L, I18H, I19L, V34Y, R39L, K46E). Although this modification improved the pharmacological profile, the in vivo effectiveness was not improved (Stassen, Thromb. Haemost. 74:655-659, 1995). In another example of PEGylated aprotinin, in which an estimated seventeen 5 kDa PEG molecules were attached, the trypsin inhibition activity was reduced by about 29-fold (Shin, Pharm. Pharmacol. Commun. 4:57-260, 1998).

Accordingly, an object of the present invention is to create novel variants of aprotinin with functional activity similar to aprotinin, especially with respect to the potency of plasmin inhibition, that exhibit improved pharmacokinetic and safety profiles and maintains in vivo efficacy.

### SUMMARY OF THE INVENTION

This invention provides novel modified variants of aprotinin that function as protease inhibitors with improved pharmacokinetic and immunogenic properties. The proteins of the present invention may be utilized, for example, to reduce blood loss during surgery, in the prevention and/or treatment of trauma, ischemia reperfusion injury, cancer, lung ciliary functions and organ transplant procedures and in applications such as fibrin glues.

In particular, one aspect of the invention is a PEGylated aprotinin selected from the group consisting of SEQ ID NOs: 3 to 15, and fragments, derivatives, and variants thereof that demonstrate at least one biological function that is substantially the same as the peptides of Table 1 (collectively, “proteins of this invention”), including functional equivalents thereof.

Another embodiment of the invention includes amino acid changes that replace residues of the bovine sequence with amino acids found in human homologues of aprotinin in order to reduce or abrogate immune recognition of aprotinin.

Another embodiment of the invention is a polynucleotide that encodes the peptides of the present invention, and the attendant vectors and host cells necessary to recombinantly express the peptides of this invention.

Another embodiment of the invention are antibodies and antibody fragments that selectively bind the peptides of this invention. Such antibodies are useful in detecting the peptides of this invention, and can be identified and made by procedures well known in the art.

### BRIEF DESCRIPTION OF THE DRAWINGS

FIG. 1. Sequence alignments of aprotinin and human Kunitz domains.

### DETAILED DESCRIPTION OF THE INVENTION

This invention provides variants of aprotinin, and fragments, derivatives, and variants thereof that demonstrate at least one biological function that is substantially the same as the proteins of Table 1 (collectively, proteins of this invention). The naturally occurring bovine aprotinin (SEQ ID NO: 1), aprotinin variants (such as SEQ ID NO: 2) and a human pharmacological equivalents such as placental bikunin (SEQ ID NO: 3) are protease inhibitors that act, for example, on trypsin, plasmin, and kallikrein. Since aprotinin usage has associated side effects such as immunogenicity, it is desirable to develop long-acting protease inhibitors that do not induce an immune response and as such would allow the possibility of repeated use of the therapeutic.

The present invention provides combinations of modifications, previously not described in the art, to manufacture aprotinin variants that are more amenable to refolding and provide for a specific PEGylation site in a benign location of aprotinin (see, e.g., Table 1, SEQ ID NO: 4-15). The peptides of this invention provide an improvement over wild-type aprotinin in terms of pharmacokinetic and immunogenicity profiles, and potentially provide beneficial therapeutic benefits without inducing other undesired safety effects such as immunogenicity, autogenicity, anaphylaxis, or renal accumulation.

One approach to improve the in vivo properties of proteins is PEGylation (Greenwald, Adv. Drug. Del. Rev. 55:217-250, 2003). To date, PEGylation has not improved the in vitro or in vivo efficacy of aprotinin. Therefore, a significant improvement over the current state-of-the-art would be attained by designing an aprotinin variant that is (i) obtained from a synthetic or recombinant source, for example, by solid-phase peptide synthesis or by expression in a prokaryotic or eukaryotic source such as Escherichia coli, yeast, baculovirus, or plants; (ii) modified to promote efficient refolding; (iii) contains a single PEGylation site that is benign in terms of moderating protease inhibition; and (iv) provides a PEG modification that improves the pharmacokinetic properties (e.g., by reducing dosing requirements) and immunogenicity.

Aprotinin may be obtained by expression in Escherichia coli (e.g., Auerswald, Biol. Chem. Hoppe Seyler 368:1413-1425, 1987; Staley, Proc. Natl. Acad. Sci. 89:1519-1523, 1992) or in transgenic plants (Azzoni, Biotechnol. Bioeng. 80:268-276, 2002) or in other expression systems such as baculovirus and yeast. Aprotinin may also be obtained by solid-phase peptide synthesis using methods known to those skilled in the art (e.g., Ferrer, Int. J. Pept. Protein Res. 40:194-207, 1992).

In addition, aprotinin variants may be produced with the recombinant approaches described above in which one or two of the three disulfide bonds of the native protein are replaced by substituting the Cys residues with another amino acid such as Ala using site-directed mutageneis (e.g., Staley, Proc. Natl. Acad. Sci. 89:1519-1523, 1992). Sequences are exemplified, but not limited by, SEQ ID NO: 4 to 6. Amino-acid changes need not necessarily be restricted to Ala. Such substitutions simplify the folding of the aprotinin variant and result in increased yield (e.g., Staley, 1992). In addition, protein disulfide isomerase may be used to increase the refolding yield (e.g., Weissman, Nature 365:185-188, 1993).

Another approach to increasing the yield of folded aprotinin is to incorporate an additional Cys residue that acts as an intramolecular catalysis of disulfide-bond formation, either as found in the native pro sequence of aprotinin (SEQ ID NO: 7) or as an unnatural amino-acid sequence (SEQ ID NO: 8) (e.g., Weissman, Cell 71:841-851, 1992). The appended sequence may be varied (e.g., SEQ ID NO: 8 and 10) and may be incorporated into aprotinin variants (e.g., SEQ ID NO: 11-14). This approach has the previously unrecognized advantage of providing a free Cys residue for site-specific modification with groups that improve pharmacokinetic properties such as polyethylene glycol (PEG).

The use of a recombinant source of aprotinin (either with or without a reduction in the number of disulfide bonds, as exemplified by combinations of SEQ ID NO: 4 to 6 with SEQ ID NO: 7 to 14) and the incorporation of an N or C terminal sequence to provide for a free Cys that both improves folding yield and provides a unique site for PEGylation offers superior manufacturing and pharmacokinetic properties over natural aprotinin isolated from bovine lung.

PEGylation may be performed by any method known to those skilled in the art. For example, PEG may be introduced to a protein by direct attachment to the N-terminal amine group, the C-terminal carboxylate group, or to an internal amino acid that contains a reactive sidechain such as Cys, Lys, Asp, or Glu, or an unnatural amino acid that contain similar reactive sidechain moieties. Numerous examples of suitable cross-linking agents are known to those skilled in the art, as exemplified by, but not limited to, commercially available PEG derivatives containing amines, aldehydes, acetals, maleimide, succinimides, and thios (e.g., Nektar Therapeutics, San Carlos, Calif., USA and NOF, Toyko, Japan).

As an example, PEGylation may be achieved by introducing a unique Cys into the peptide via a N-terminal or C-terminal modifying amino-acid sequence that does not form a disulfide bond with one of the six naturally occurring Cys residues of aprotinin after refolding. The unique Cys is then PEGylated via a stable thioether linkage between the mercapto group and maleimide group of methoxy-PEG-maleimide reagents (e.g., Nektar Therapeutics, San Carlos, Calif., USA, and/or NOF, Tokyo, Japan). In addition to maleimide, numerous Cys reactive groups are known to those skilled in the art of protein cross-linking, such as the use of alkyl halides and vinyl sulfones.

Various size PEG groups may be used as exemplified, but not limited to, PEG polymers of from about 5 kDa to about 43 kDa. The PEG modification may include a single, linear PEG, such as linear 5, 20, or 30 kDa PEGs attached to maleidmide or other cross-linking groups (see, e.g., Table 2). Also, the modification may involve branched PEGs that contain two or more PEG polymer chains attached to maleimide or other cross-linking groups (see, e.g., Table 2).

PEGylation with a smaller PEG (e.g., a linear 5 kDa PEG) is less likely to reduce the activity of the peptide, whereas a larger PEG (e.g., a branched 40 kDa PEG) is more likely to reduce activity. However, a larger PEG will increase plasma half-life so that a reduced dose is possible.

The linker between the PEG and the cross-linking group of the PEG reagent may be varied. For example, the commercially available Cys-reactive 40 kDa PEG (mPEG2-MAL) from Nektar Therapeutics (San Carlos, Calif., USA) employs a maleimide group for conjugation to Cys, and the maleimide group is attached to the PEG via a linker based on lysine (Table 2). As a second example, the commercially available Cys-reactive 43 kDa PEG (GL2-400MA) from NOF (Toyko, Japan) employs a maleimide group for conjugation to Cys, and the maleimide group is attached to the PEG via a bisubstituted alkane linker (Table 2). In addition, the PEG polymer can be attached directly to the maleimide, as exemplified by PEG reagents of molecular weight 5 kDa and 20 kDa available form Nektar Therapeutics (San Carlos, Calif., USA) (Table 2).

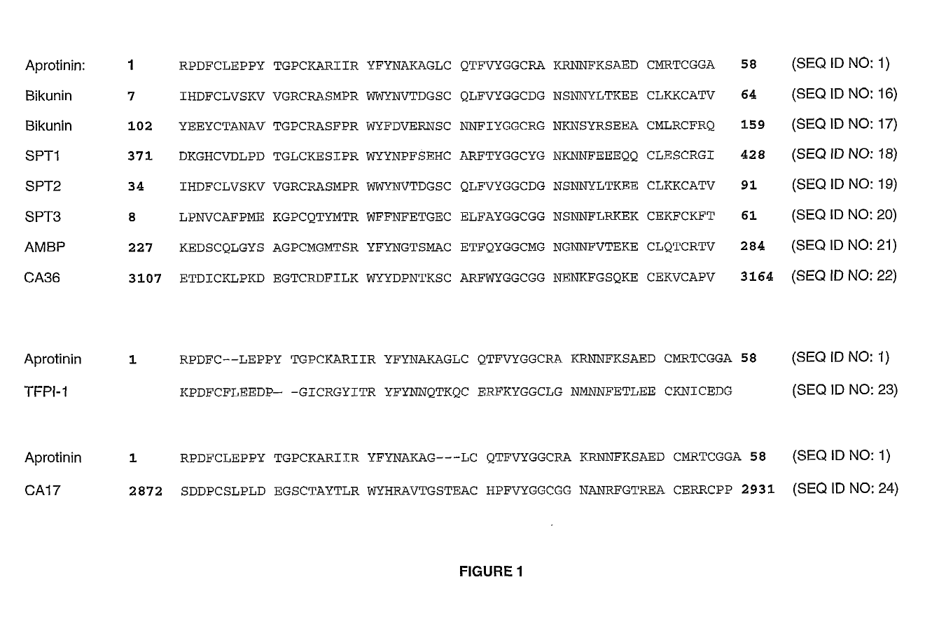

In addition to PEGylation, another approach to improving the pharmacokinetic, immunogenetic, or other safety properties of a protein is the use of amino acid replacements. Since the immune system will not normally recognize endogenous protein sequences, aprotinin variants include those in which residues that differ from human homologues are replaced with the corresponding amino acid of the human protein. Such variants would preferably target surface-exposed amino acids, which are identified using the known atomic-resolution structures of aprotinin, and would involve substituting the amino acid of the bovine protein with that found in a human homologue such as a Kunitz domain of human placental bikunin (FIG. 1). Such variants could also include amino acid changes of buried or partially buried residues. One or more amino acid substitutions or whole-domain swaps can be made in aprotinin to produce a sequence that is similar to human homologues as exemplified by, but not limited to, SEQ ID NO: 10 to 13 (Table 1). Changes are based upon sequence alignments between aprotinin and human homologues. For example, Arg 1 of aprotinin may be replaced with Ile 7 or Tyr 102 of human placental bikunin, or Pro 3 of aprotinin may be replaced with His 8 or Glu 103 of human placental bikunin (FIG. 1). One skilled in the art can readily identify such changes from sequence alignments as exemplified by, but not limited to, those of FIG. 1, and one or more such changes may be incorporated into a single aprotinin variant.

The variations to aprotinin described above are exemplified by the following sequence:

(SEQ ID NO: 15)

A₁A₂A₃A₄A₅A₆A₇A₈A₉A₁₀ RPDFC₅LEPPY TGPC₁₄KARIIR
YFYNAKAGLC₃₀ QTFVYGGC₃₈RA KRNNFKSAED C₅₁MRTC₅₅GG
A₁₁A₁₂A₁₃A₁₄A₁₅A₁₆A₁₇A₁₈A₁₉A₂₀

wherein

A₁ to A₂₀ may be naturally occurring amino acids, unnatural amino acids, or deleted, and wherein at least one residue (A₁ to A₂₀) is cysteine (Cys). As an example, A₁ to A₂₀ may be lysine, glutamine, asparagine, serine, threonine, glycine, alanine, or cysteine. In addition, to reduce the number of disulfide bonds required for folding, the following pairs of cysteines: C₅ and C₅₅, C₁₄ and C₃₈, or C₃₀ and C₅₁, may be substituted to alanine, and wherein one pair of cysteines is not substituted. Furthermore, as exemplified in SEQ ID NO: 15, the N- and C-terminal additions (A₁ to A₁₀ and A₁₁ to A₂₀, respectively) may be greater than ten residues.

In addition to PEGylation, other polymers derivatized with Cys-reactive groups may be employed to improve the pharmacokinetic or immunogenic properties of aprotinin variants. For example, but not by way of limitation, aprotinin variants may be modified with hydroxyethylstarch (e.g., WO 2004/024761).

It will be recognized that the invention described here as relating to aprotinin variants provides an approach to manufacturing other PEGylated proteins that contain disulfide bonds such as human protease inhibitors containing Kunitz domains.

Certain terms used throughout this specification will now be defined, and others will be defined as introduced. The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (lie); K, lycine (Lys); L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); and Y, tyrosine (Tyr).

The term “polynucleotide encoding a peptide” encompasses a polynucleotide which includes only coding sequence for the peptide, as well as a polynucleotide which includes additional coding and/or non-coding sequence. The present invention further relates to polynucleotides which hybridize to the hereinabove-described sequences if there is at least about 70%, at least about 90%, and at least about 95% identity between the sequences. The present invention particularly relates to polynucleotides encoding peptides which hybridize under stringent conditions to the hereinabove-described polynucleotides. As herein used, the term “stringent conditions” means “stringent hybridization conditions.” Hybridization may occur only if there is at least about 90% or about 95% through 97% identity between the sequences. The polynucleotides which hybridize to the hereinabove described polynucleotides in one embodiment encode peptides which retain substantially the same biological function or activity as the mature peptide encoded by the cDNAs.

“Functional equivalent” and “substantially the same biological function or activity” each means that degree of biological activity that is within about 30% to about 100% or more of that biological activity demonstrated by the peptide to which it is being compared when the biological activity of each peptide is determined by the same procedure.

The terms “fragment,” “derivative,” and “variant,” when referring to the peptides of the present invention, means fragments, derivatives, and variants of the peptides which retain substantially the same biological function or activity as such peptides, as described further below.

A fragment is a portion of the peptide which retains substantially similar functional activity, as described in the in vivo models disclosed herein.

A derivative includes all modifications to the peptide which substantially preserve the functions disclosed herein and include additional structure and attendant function (e.g., modified N-terminus peptides, PEGylated peptides), fusion peptides which confer targeting specificity or an additional activity such as toxicity to an intended target, as described further below.

The peptides of the present invention may be recombinant peptides, natural purified peptides, or synthetic peptides.

The fragment, derivative, or variant of the peptides of the present invention may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature peptide is fused with another compound, such as a compound to increase the half-life of the peptide, or (iv) one in which the additional amino acids are fused to the mature peptide, such as a leader or secretory sequence or a sequence which is employed for purification of the mature peptide, or (v) one in which the peptide sequence is fused with a larger peptide (e.g., human albumin, an antibody or Fc, for increased duration of effect). Such fragments, derivatives, and variants and analogs are deemed to be within the scope of those skilled in the art from the teachings herein.

The derivatives of the present invention may contain conservative amino acid substitutions (defined further below) made at one or more nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Fragments, or biologically active portions include peptide fragments suitable for use as a medicament, to generate antibodies, as a research reagent, and the like. Fragments include peptides comprising amino acid sequences sufficiently similar to or derived from the amino acid sequences of a peptide of this invention and exhibiting at least one activity of that peptide, but which include fewer amino acids than the full-length peptides disclosed herein. Typically, biologically active portions comprise a domain or motif with at least one activity of the peptide. A biologically active portion of a peptide can be a peptide which is, for example, five or more amino acids in length. Such biologically active portions can be prepared synthetically or by recombinant techniques and can be evaluated for one or more of the functional activities of a peptide of this invention by means disclosed herein and/or well known in the art.

Moreover, derivatives of the present invention may include peptides that have been fused with another compound, such as a compound to increase the half-life of the peptide and/or to reduce potential immunogenicity of the peptide (e.g., polyethylene glycol, “PEG”). In the case of PEGylation, the fusion of the peptide to PEG can be accomplished by any means known to one skilled in the art. For example, PEGylation can be accomplished by first introducing a cysteine mutation into the peptide to provide a linker upon which to attach the PEG, followed by site-specific derivatization with PEG-maleimide. For example, the cysteine can be added to the C-terminus of the peptides. (see, e.g., Tsutsumi, et al., Proc. Natl. Acad. Sci. USA 97(15):8548-53, 2000; Veronese, Biomaterials 22:405-417, 2001; Goodsoon & Katre, Bio/Technology 8:343-346, 1990). Variants of the peptides of this invention include peptides having an amino acid sequence sufficiently similar to the amino acid sequence of the peptides of this invention or a domain thereof. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, about 75% through 98%, identical are defined herein as sufficiently similar. Variants will be sufficiently similar to the amino acid sequence of the peptides of this invention. Variants include variants of peptides encoded by a polynucleotide that hybridizes to a polynucleotide of this invention or a complement thereof under stringent conditions. Such variants generally retain the functional activity of the peptides of this invention. Libraries of fragments of the polynucleotides can be used to generate a variegated population of fragments for screening and subsequent selection. For example, a library of fragments can be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the peptide of this invention.

Variants include peptides that differ in amino acid sequence due to mutagenesis. Variants that function as aprotinin can be identified by screening combinatorial libraries of mutants, for example truncation mutants, of the peptides of this invention for aprotinin activity.

In one embodiment, a variegated library of analogs is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential variant amino acid sequences is expressible as individual peptides, or, alternatively, as a set of larger fusion proteins (for example, for phage display) containing the set of sequences therein. There are a variety of methods that can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential variant sequences. Methods for synthesizing degenerate oligonucleotides are known in the art (see, e.g., Narang, Tetrahedron 39:3, 1983; Itakura, et al., Annu. Rev. Biochem. 53:323,1984; Itakura, et al., Science 198:1056, 1984; Ike, et al., Nucleic Acid Res. 11:477, 1983).

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of R-agonist peptides. The most widely used techniques, which are amenable to high through-put analysis for screening large gene libraries typically include cloning the gene library into replicable expression vectors, transforming appropriate cells with the resulting library of vectors and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify the desired variants.

The peptides of this invention can be composed of amino acids joined to each other by peptide bonds or modified peptide bonds (i.e., peptide isosteres), and may contain amino acids other than the 20 gene-encoded amino acids. The peptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a peptide, including the peptide backbone, the amino acid side-chains and the amino or carboxyl termini. It will be appreciated that the same type of modification may be present in the same or varying degrees at several sites in a given peptide. Also, a given peptide may contain many types of modifications. Peptides may be branched, for example, as a result of ubiquitination, and they may be cyclic, with or without branching. Cyclic, branched, and branched cyclic peptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Proteins, Structure and Molecular Properties, 2nd ed., T. E. Creighton, W. H. Freeman and Company, New York (1993); Posttranslational Covalent Modification of Proteins, B. C. Johnson, ed., Academic Press, New York, pgs. 1-12 (1983); Seifter, et al., Meth. Enzymol 182:626-646, 1990; Rattan, et al., Ann. N.Y. Acad. Sci. 663:48-62, 1992).

The peptides of the present invention include the peptides of SEQ ID NOs: 3 through 15, as well as those sequences having insubstantial variations in sequence from them. An “insubstantial variation” would include any sequence addition, substitution, or deletion variant that maintains substantially at least one biological function of the peptides of this invention, for example, aprotinin activity. These functional equivalents may include peptides which have at least about 70% identity to the peptides of the present invention, at least 90% identity to the peptides of the present invention, and at least 95% identity to the peptides of the present invention, and also include portions of such peptides having substantially the same biological activity. However, any peptide having insubstantial variation in amino acid sequence from the peptides of the present invention that demonstrates functional equivalency as described further herein is included in the description of the present invention.

As known in the art “similarity” between two peptides is determined by comparing the amino acid sequence and the conserved amino acid substitutes of one peptide to the sequence of a second peptide. Such conservative substitutions include those described above and by Dayhoff (The Atlas of Protein Sequence and Structure 5, 1978), and by Argos (EMBO J. 8:779-785, 1989). For example, amino acids belonging to one of the following groups represent conservative changes:
Ala, Pro, Gly, Gln, Asn, Ser, Thr;
Cys, Ser, Tyr, Thr;
Val, Ile, Leu, Met, Ala, Phe;
Lys, Arg, His;
Phe, Tyr, Trp, His; and
Asp, Glu.

The present invention also relates to polynucleotides encoding the peptides of this invention, as well as vectors which include these polynucleotides, host cells which are genetically engineered with vectors of the invention, and the production of peptides of the invention by recombinant techniques. Host cells may be genetically engineered (transduced, transformed, or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, or selecting transformants. The culture conditions, such as temperature, pH and the like, are those previously used with the host cell selected for expression, and will be apparent to the ordinarily skilled artisan. The polynucleotide of the present invention may be employed for producing a peptide by recombinant techniques. Thus, for example, the polynucleotide sequence may be included in any one of a variety of expression vehicles, in particular, vectors or plasmids for expressing a peptide. Such vectors include chromosomal, non-chromosomal, and synthetic DNA sequences (e.g., derivatives of SV40); bacterial plasmids; phage DNA; yeast plasmids; vectors derived from combinations of plasmids and phage DNA; viral DNA such as vaccinia, adenovirus, fowl pox virus, and pseudorabies. However, any other vector or plasmid may be used as long as they are replicable and viable in the host.

The appropriate DNA sequence may be inserted into the vector by a variety of procedures. In general, the DNA sequence is inserted into an appropriate restriction endonuclease site by procedures known in the art. Such procedures and others are deemed to be within the scope of those skilled in the art. The DNA sequence in the expression vector is operatively linked to an appropriate expression control sequence(s) (promoter) to direct mRNA synthesis. Representative examples of such promoters include, but are not limited to, LTR or SV40 promoter, the E. coli lac, T7, or trp, the phage lambda PL promoter, and other promoters known to control expression of genes in prokaryotic or eukaryotic cells or their viruses. The expression vector may also contain a ribosome binding site for translation initiation and a transcription terminator. The vector may also include appropriate sequences for amplifying expression. In addition, the expression vectors may contain a gene to provide a phenotypic trait for selection of transformed host cells such as dihydrofolate reductase or neomycin resistance for eukaryotic cell culture, or such as tetracycline or ampicillin resistance in E. coli. The vector containing the appropriate DNA sequence as herein above described, as well as an appropriate promoter or control sequence, may be employed to transform an appropriate host to permit the host to express the protein. Representative examples of appropriate hosts, include, but are not limited to, bacterial cells, such as E. coli, Salmonella typhimurium, Streptomyces; fungal cells, such as yeast; insect cells, such as Drosophila S2 and Spodoptera Sf9; animal cells such as CHO, COS, or Bowes melanoma; adenoviruses; plant cells, etc. The selection of an appropriate host is deemed to be within the scope of those skilled in the art from the teachings herein.

The present invention also includes recombinant constructs comprising one or more of the sequences as broadly described above. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example. Bacterial: pET vectors, pQE70, pQE60, pQE-9, pBS, phagescript, psiX174, pBluescript SK, pBsKS, pNH8a, pNH16a, pNH18a, pNH46a, pTRC99A, pKK223-3, pKK233-3, pDR540, and PRIT5. Eukaryotic: pWLneo, pSV2cat, pOG44, pXT1, pSG, pSVK3, pBPV, pMSG, and PSVL. However, any other plasmid or vector may be used as long as they are replicable and viable in the host. Promoter regions can be selected from any desired gene using CAT(chloramphenicol transferase) vectors or other vectors with selectable markers. Two appropriate vectors are pKK232-8 and pCM7. Particular named bacterial promoters include laci, lacZ, T3, T7, gpt, lambda P_{R}, P_{L}, and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The present invention also relates to host cells containing the above-described construct. The host cell can be a higher eukaryotic cell such as a mammalian cell or a lower eukaryotic cell such as a yeast cell, or the host cell can be a prokaryotic cell such as a bacterial cell. Introduction of the construct into the host cell can be effected by calcium phosphate transfection, DEAE-Dextran mediated transfection, or electroporation (Davis, et al., Basic Methods in Molecular Biology, 1986). The constructs in host cells can be used in a conventional manner to produce the gene product encoded by the recombinant sequence. Alternatively, the peptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, (Cold Spring Harbor, N.Y., 1989), the disclosure of which is hereby incorporated by reference.

Transcription of a DNA encoding the peptides of the present invention by higher eukaryotes is increased by inserting an enhancer sequence into the vector. Enhancers are cis-acting elements of DNA, usually from about 10 to about 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Generally, recombinant expression vectors will include origins of replication and selectable markers permitting transformation of the host cell (e.g., the ampicillin resistance gene of E. coli or S. cerevisiae TRP1 gene), and a promoter derived from a highly-expressed gene to direct transcription of a downstream structural sequence. Such promoters can be derived from operons encoding glycolytic enzymes such as 3-phosphoglycerate kinase (PGK), α factor, acid phosphatase, or heat shock proteins, among others. The heterologous structural sequence is assembled in appropriate phase with translation, initiation and termination sequences, and optionally a leader sequence capable of directing secretion of translated protein into the periplasmic space or extracellular medium. Optionally, the heterologous sequence can encode a fusion protein including an N-terminal identification peptide imparting desired characteristics (e.g., stabilization or simplified purification of expressed recombinant product).

Useful expression vectors for bacterial use may be constructed by inserting a structural DNA sequence encoding a desired protein together with suitable translation, initiation, and termination signals in operable reading phase with a functional promoter. The vector may comprise one or more phenotypic selectable markers and an origin of replication to ensure maintenance of the vector and to, if desirable, provide amplification within the host. Suitable prokaryotic hosts for transformation include, for example, E. coli, Bacillus subtilis, Salmonella typhimurium, and various species within the genera Pseudomonas, Streptomyces, and Staphylococcus, although others may also be employed as a matter of choice. Useful expression vectors for bacterial use may comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega, Madison, Wis., USA). These pBR322 “backbone” sections may be combined with an appropriate promoter and the structural sequence to be expressed.

After transformation of a suitable host strain and growth of the host strain to an appropriate cell density, the selected promoter is derepressed by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents.

Various mammalian cell culture systems may also be employed to express recombinant protein. Examples of mammalian expression systems include the COS-7 lines of monkey kidney fibroblasts described by Gluzman, (Cell 23:175, 1981), and other cell lines capable of expressing a compatible vector, for example, the C127, 3T3, CHO, HeLa, and BHK cell lines. Mammalian expression vectors may comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required non-transcribed genetic elements.

The peptides of the present invention may be recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. Protein refolding steps can be used, as necessary, in completing configuration of the mature protein. Finally, high performance liquid chromatography (HPLC) may be employed for final purification steps.

The peptides of this invention may be a product of chemical synthetic procedures, or produced by recombinant techniques from a prokaryotic or eukaryotic host (e.g., bacterial, yeast, higher plant, insect, and mammalian cells). Depending upon the host employed in a recombinant production procedure, the peptides of this invention may be glycosylated with mammalian or other eukaryotic carbohydrates, or may be non-glycosylated. Peptides of this invention may also include an initial methionine amino acid residue. An isolated or purified peptide of this invention, or biologically active portion thereof, is substantially free of other cellular material, or culture medium when produced by recombinant techniques, or substantially free of chemical precursors or other chemicals when chemically synthesized. An isolated peptide of this invention is substantially free of cellular material and has less than about 30% (by dry weight) of non-peptide, or contaminating, material. When the peptide of this invention or a biologically active portion thereof is recombinantly produced, culture medium may represent less than about 30% of the volume of the peptide preparation. When this invention is produced by chemical synthesis, the preparations may contain less than about 30% by dry weight of chemical precursors or non-invention chemicals.

The peptides of this invention may be conveniently isolated as described in the specific examples below. A preparation of purified peptide is at least about 70% pure; or about 85% through about 99% pure. Purity of the preparations can be assessed by any means known in the art, such as SDS-polyacrylamide gel electrophoresis and Mass Spec/Liquid Chromatography.

Polynucleotide sequences encoding a peptide of this invention may be synthesized, in whole or in part, using chemical methods well known in the art (see, e.g., Caruthers, et al., Nucl. Acids Res. Symp. Ser. 215-223, 1980; Horn, et al., Nucl. Acids Res. Symp. Ser. 225-232, 1980). The polynucleotide that encodes the peptide may then be cloned into an expression vector to express the peptide.

As will be understood by those of skill in the art, it may be advantageous to produce the peptide-encoding nucleotide sequences possessing non-naturally occurring codons. For example, codons preferred by a particular prokaryotic or eukaryotic host can be selected to increase the rate of peptide expression or to produce an RNA transcript having desirable properties, such as a half-life which is longer than that of a transcript generated from the naturally occurring sequence.

The nucleotide sequences disclosed herein may be engineered using methods generally known in the art to alter the peptide-encoding sequences for a variety of reasons, including but not limited to, alterations which modify the closing, processing, and/or expression of the peptide or mRNA product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

Also provided are related peptides within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a peptide of the present invention. It will be understood that the phrase “equivalent to” as used herein is intended to encompass peptides having substitution(s) of certain atoms, or chemical moieties in said peptide, having bond lengths, bond angles, and arrangements in the mimetic peptide that produce the same or sufficiently similar arrangement or orientation of said atoms and moieties to have the biological function of the peptides of the invention. In the peptide mimetics of the invention, the three-dimensional arrangement of the chemical constituents is structurally and/or functionally equivalent to the three-dimensional arrangement of the peptide backbone and component amino acid sidechains in the peptide, resulting in such peptido-, organo-, and chemical mimetics of the peptides of the invention having substantial biological activity. These terms are used according to the understanding in the art, as illustrated, for example, by Fauchere, (Adv. Drug Res. 15:29, 1986); Veber & Freidinger, (TINS p.392, 1985); and Evans, et al., (J. Med. Chem. 30:1229, 1987), incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo-, and chemical mimetics may be designed to fit each pharmacophore with current computer modeling software (computer aided drug design). Said mimetics may be produced by structure-function analysis, based on the positional information from the substituent atoms in the peptides of the invention.

Peptides as provided by the invention can be advantageously synthesized by any of the chemical synthesis techniques known in the art, particularly solid-phase synthesis techniques, for example, using commercially-available automated peptide synthesizers. The mimetics of the present invention can be synthesized by solid phase or solution phase methods conventionally used for the synthesis of peptides (see, e.g., Merrifield, J. Amer. Chem. Soc. 85:2149-54, 1963; Carpino, Acc. Chem. Res. 6:191-98, 1973; Birr, Aspects of the Merrifield Peptide Synthesis, Springer-Verlag: Heidelberg, 1978; The Peptides: Analysis, Synthesis, Biology, Vols. 1, 2, 3, and 5, (Gross & Meinhofer, eds.), Academic Press: New York, 1979; Stewart, et al., Solid Phase Peptide Synthesis, 2nd. ed., Pierce Chem. Co.: Rockford, Ill., 1984; Kent, Ann. Rev. Biochem. 57:957-89, 1988; and Gregg, et al., Int. J. Peptide Protein Res. 55:161-214, 1990, which are incorporated herein by reference in their entirety.)

Peptides of the present invention may be prepared by solid phase methodology. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods, or the like. Cycles of deprotection, neutralization, and coupling of successive protected amino acid derivatives are used to link the amino acids from the C-terminus according to the amino acid sequence. For some synthetic peptides, an FMOC strategy using an acid-sensitive resin may be used. Solid supports in this regard may be divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). A protecting group for alpha amino acids is base-labile 9-fluorenylmethoxy-carbonyl (FMOC).

Suitable protecting groups for the side chain functionalities of amino acids chemically compatible with BOC (t-butyloxycarbonyl) and FMOC groups are well known in the art. The amino acid residues may be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexa-fluorophosphate), PyBOP (benzotriazole-1-yl-oxy-tris-pyrrolidinophosphonium hexafluoro-phosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-1-yl),1,1,3,3-tetramethyluronium hexafluoro-phosphate) in the presence of HOBt or HOAt (7-azahydroxybenztriazole) is preferred.

The solid phase method may be carried out manually, and automated synthesis on a commercially available peptide synthesizer (e.g., Applied Biosystems 433A or the like; Applied Biosystems, Foster City, Calif.) is also available. In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (Applied Biosystems) may be used to generate the peptide sequence. Double and triple coupling, with capping by acetic anhydride, may also be used.

The synthetic mimetic peptide may be cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, may be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization of the appropriate base.

Generally, those skilled in the art will recognize that peptides as described herein may be modified by a variety of chemical techniques to produce peptides having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric, and other organic salts, or may be converted to an amide. Those skilled in the art will also recognize methods for introducing cyclic structures into the peptides of this invention so that the native binding configuration will be more nearly approximated.

A variety of techniques are available for constructing peptide derivatives and analogs with the same or similar desired biological activity as the corresponding peptide but with more favorable activity than the peptide with respect to solubility, stability, and susceptibility to hydrolysis and proteolysis. Such derivatives and analogs include peptides modified at the N-terminal amino group, the C-terminal carboxyl group, and/or changing one or more of the amido linkages in the peptide to a non-amido linkage. It will be understood that two or more such modifications may be coupled in one peptide mimetic structure (e.g., modification at the C-terminal carboxyl group and inclusion of a —CH₂— carbamate linkage between two amino acids in the peptide).

Amino terminus modifications include alkylating, acetylating, adding a carbobenzoyl group, and forming a succinimide group. Specifically, the N-terminal amino group may be reacted to form an amide group of the formula RC(O)NH— where R is alkyl, and is added by reaction with an acid halide, RC(O)Cl or acid anhydride. Typically, the reaction can be conducted by contacting about equimolar or excess amounts (e.g., about 5 equivalents) of an acid halide to the peptide in an inert diluent (e.g., dichloromethane) containing an excess (e.g., about 10 equivalents) of a tertiary amine, such as diisopropylethylamine, to scavenge the acid generated during reaction. Reaction conditions are otherwise conventional (e.g., room temperature for 30 minutes). Alkylation of the terminal amino to provide for a lower alkyl N-substitution followed by reaction with an acid halide as described above will provide an N-alkyl amide group of the formula RC(O)NR—. Alternatively, the amino terminus may be covalently linked to succinimide group by reaction with succinic anhydride. An approximately equimolar amount or an excess of succinic anhydride (e.g., about 5 equivalents) is used and the terminal amino group is converted to the succinimide by methods well known in the art including the use of an excess (e.g., 10 equivalents) of a tertiary amine such as diisopropylethylamine in a suitable inert solvent (e.g., dichloromethane), as described in Wollenberg, et al., (U.S. Pat. No. 4,612,132), and is incorporated herein by reference in its entirety. It will also be understood that the succinic group may be substituted with, for example, a C₂- through C₆-alkyl or —SR substituents, which are prepared in a conventional manner to provide for substituted succinimide at the N-terminus of the peptide. Such alkyl substituents may be prepared by reaction of a lower olefin (C₂- through C6-alkyl) with maleic anhydride in the manner described by Wollenberg, et al., supra., and —SR substituents may be prepared by reaction of RSH with maleic anhydride where R is as defined above. In another advantageous embodiment, the amino terminus may be derivatized to form a benzyloxycarbonyl-NH— or a substituted benzyloxycarbonyl-NH— group. This derivative may be produced by reaction with approximately an equivalent amount or an excess of benzyloxycarbonyl chloride (CBZ-Cl), or a substituted CBZ-Cl in a suitable inert diluent (e.g., dichloromethane) containing a tertiary amine to scavenge the acid generated during the reaction. In yet another derivative, the N-terminus comprises a sulfonamide group by reaction with an equivalent amount or an excess (e.g., 5 equivalents) of R—S(O)₂Cl in a suitable inert diluent (dichloromethane) to convert the terminal amine into a sulfonamide, where R is alkyl (e.g., lower alkyl). The inert diluent contains excess tertiary amine (e.g., 10 equivalents) such as diisopropylethylamine, to scavenge the acid generated during reaction. Reaction conditions are otherwise conventional (e.g., room temperature for 30 minutes). Carbamate groups may be produced at the amino terminus by reaction with an equivalent amount or an excess (e.g., 5 equivalents) of R—OC(O)Cl or R—OC(O)OC₆H₄—p—NO₂ in a suitable inert diluent (e.g., dichloromethane) to convert the terminal amine into a carbamate, where R is alkyl (e.g., lower alkyl). The inert diluent may contain an excess (e.g., about 10 equivalents) of a tertiary amine, such as diisopropylethylamine, to scavenge any acid generated during reaction. Reaction conditions are otherwise conventional (e.g., room temperature for 30 minutes). Urea groups may be formed at the amino terminus by reaction with an equivalent amount or an excess (e.g., 5 equivalents) of R—N═C═O in a suitable inert diluent (e.g., dichloromethane) to convert the terminal amine into a urea (i.e., RNHC(O)NH—) group where R is as defined above. The inert diluent may contain an excess (e.g., about 10 equivalents) of a tertiary amine, such as diisopropylethylamine. Reaction conditions are otherwise conventional (e.g., room temperature for about 30 minutes).

In preparing peptide mimetics wherein the C-terminal carboxyl group may be replaced by an ester (e.g., —C(O)OR where R is alkyl), resins used to prepare the peptide acids may be employed, and the side chain protected peptide may be cleaved with a base and the appropriate alcohol (e.g., methanol). Side chain protecting groups may be removed in the usual fashion by treatment with hydrogen fluoride to obtain the desired ester. In preparing peptide mimetics wherein the C-terminal carboxyl group is replaced by the amide —C(O)NR₃R₄, a benzhydrylamine resin is used as the solid support for peptide synthesis. Upon completion of the synthesis, hydrogen fluoride treatment to release the peptide from the support results directly in the free peptide amide (i.e., the C-terminus is —C(O)NH₂). Alternatively, use of the chloromethylated resin during peptide synthesis coupled with reaction with ammonia to cleave the side chain protected peptide from the support yields the free peptide amide, and reaction with an alkylamine or a dialkylamine yields a side chain protected alkylamide or dialkylamide (i.e., the C-terminus is —C(O)NRR₁, where R and R₁ are alkyl, a lower alkyl). Side chain protection is then removed in the usual fashion by treatment with hydrogen fluoride to give the free amides, alkylamides, or dialkylamides.

Peptide mimetics as understood in the art and provided by the invention are structurally similar to the peptide of the invention, but have one or more peptide linkages optionally replaced by a linkage selected from the group consisting of: —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, Chemistry and Biochemistry of Amino Acids, Peptides, and Proteins, (Weinstein, ed.), Marcel Dekker: New York, p. 267, 1983; Spatola, Peptide Backbone Modifications 1:3,1983; Morley, Trends Pharm. Sci. pp. 463-468, 1980; Hudson, et al., Int. J. Pept. Prot. Res. 14:177-185, 1979; Spatola, et al., Life Sci. 38:1243-1249, 1986; Hann, J. Chem. Soc. Perkin Trans. I 307-314, 1982; Almquist, et al., J. Med. Chem. 23:1392-1398, 1980; Jennings-White, et al., Tetrahedron Left. 23:2533, 1982; Szelke, et al., EP045665A; Holladay, et al., Tetrahedron Lett. 24:4401-4404, 1983; and Hruby, Life Sci. 31:189-199, 1982; each of which is incorporated herein by reference. Such peptide mimetics may have significant advantages over peptide embodiments, including, for example, more economical to produce, having greater chemical stability or enhanced pharmacological properties (such as half-life, absorption, potency, efficacy, etc.), reduced antigenicity, and other properties.

Mimetic analogs of the peptides of the invention may also be obtained using the principles of conventional or rational drug design (see, e.g., Andrews, et al., Proc. Alfred Benzon Symp. 28:145-165, 1990; McPherson, Eur. J. Biochem. 189:1-24, 1990; Hol, et al., in Molecular Recognition: Chemical and Biochemical Problems, (Roberts, ed.); Royal Society of Chemistry; pp. 84-93, 1989a; Hol, Arzneim-Forsch. 39:1016-1018, 1989b; Hol, Agnew Chem. Int. Ed. Engl. 25:767-778, 1986; the disclosures of which are herein incorporated by reference).

In accordance with the methods of conventional drug design, the desired mimetic molecules may be obtained by randomly testing molecules whose structures have an attribute in common with the structure of a “native” peptide. The quantitative contribution that results from a change in a particular group of a binding molecule may be determined by measuring the biological activity of the putative mimetic in comparison with the activity of the peptide. In one embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

One method for performing rational mimetic design employs molecular graphics software capable of forming a representation of the three-dimensional structure of the peptide. Molecular structures of the peptido-, organo-, and chemical mimetics of the peptides of the invention may be produced using computer-assisted design programs commercially available in the art. Examples of such programs include SYBYL 6.5®, HQSAR™, and ALCHEMY 2000™ (Tripos); GALAXY™ and AM200™ (AM Technologies, Inc., San Antonio, Tex.); CATALYST™ and CERIUS™ (Molecular Simulations, Inc., San Diego, Calif.); CACHE PRODUCTS™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo-, and chemical mimetics produced using the peptides disclosed herein using, for example, art-recognized molecular modeling programs may be produced using conventional chemical synthetic techniques, for example, designed to accommodate high throughput screening, including combinatorial chemistry methods. Combinatorial methods useful in the production of the peptido-, organo-, and chemical mimetics of the invention include phage display arrays, solid-phase synthesis, and combinatorial chemistry arrays, as provided, for example, by SIDDCO (Tuscon, Ariz.); Tripos, Inc.; Calbiochem/Novabiochem (San Diego, Calif.); Symyx Technologies, Inc. (Santa Clara, Calif.); Medichem Research, Inc. (Lemont, Ill.); Pharm-Eco Laboratories, Inc. (Bethlehem, Pa.); or N.V. Organon (Oss, Netherlands). Combinatorial chemistry production of the peptido-, organo-, and chemical mimetics of the invention may be produced according to methods known in the art, including, but not limited to, techniques disclosed in Terrett, (Combinatorial Chemistry, Oxford University Press, London, 1998); Gallop, et al., J. Med. Chem. 37:1233-51, 1994; Gordon, et al., J. Med. Chem. 37:1385-1401, 1994; Look, et al., Bioorg. Med. Chem. Lett. 6:707-12, 1996; Ruhland, et al., J. Amer. Chem. Soc. 118: 253-4, 1996; Gordon, et al., Acc. Chem. Res. 29:144-54, 1996; Thompson & Ellman, Chem. Rev. 96:555-600, 1996; Fruchtel & Jung, Angew. Chem. Int. Ed. Engl. 35:17-42, 1996; Pavia, “The Chemical Generation of Molecular Diversity”, Network Science Center, www.netsci.org, 1995; Adnan, et al., “Solid Support Combinatorial Chemistry in Lead Discovery and SAR Optimization,” Id., 1995; Davies and Briant, “Combinatorial Chemistry Library Design using Pharmacophore Diversity,” Id., 1995; Pavia, “Chemically Generated Screening Libraries: Present and Future,” Id., 1996; and U.S. Pat. Nos. 5,880,972; 5,463,564; 5,331573; and 5,573,905.

The newly synthesized peptides may be substantially purified by preparative high performance liquid chromatography (see, e.g., Creighton, Proteins: Structures And Molecular Principles, WH Freeman and Co., New York, N.Y., 1983). The composition of a synthetic peptide of the present invention may be confirmed by amino acid analysis or sequencing by, for example, the Edman degradation procedure (Creighton, supra). Additionally, any portion of the amino acid sequence of the peptide may be altered during direct synthesis and/or combined using chemical methods with sequences from other proteins to produce a variant peptide or a fusion peptide.

Also included in this invention are antibodies and antibody fragments that selectively bind the peptides of this invention. Any type of antibody known in the art may be generated using methods well known in the art. For example, an antibody may be generated to bind specifically to an epitope of a peptide of this invention. “Antibody” as used herein includes intact immunoglobulin molecules, as well as fragments thereof, such as Fab, F(ab′)₂, and Fv, which are capable of binding an epitope of a peptide of this invention. Typically, at least 6, 8, 10, or 12 contiguous amino acids are required to form an epitope. However, epitopes which involve non-contiguous amino acids may require more amino acids, for example, at least 15, 25, or 50 amino acids.

An antibody which specifically binds to an epitope of a peptide of this invention may be used therapeutically, as well as in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a peptide of this invention provides a detection signal at least 5-, 10-, or 20-fold higher than a detection signal provided with other proteins when used in an immunochemical assay. Preferably, antibodies which specifically bind to a peptide of this invention do not detect other proteins in immunochemical assays and can immunoprecipitate a peptide of this invention from solution.

Peptides of this invention may be used to immunize a mammal, such as a mouse, rat, rabbit, guinea pig, monkey, or human, to produce polyclonal antibodies. If desired, a peptide of this invention may be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a peptide of this invention may be prepared using any technique which provides for the production of antibody molecules by continuous cell lines in culture. These techniques include, but are not limited to, the hybridoma technique, the human B cell hybridoma technique, and the EBV hybridoma technique (Kohler, et al., Nature 256:495-97, 1985; Kozbor, et al., J. Immunol. Methods 81:3142, 1985; Cote, et al., Proc. Natl. Acad. Sci. 80:2026-30, 1983; Cole, et al., Mol. Cell Biol. 62:109-20, 1984).

In addition, techniques developed for the production of “chimeric antibodies,” the splicing of mouse antibody genes to human antibody genes to obtain a molecule with appropriate antigen specificity and biological activity, may be used (Morrison, et al., Proc. Natl. Acad. Sci. 81:6851-55, 1984; Neuberger, et al., Nature 312:604-08, 1984; Takeda, et al., Nature 314:452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences may be minimized by replacing residues which differ from those in the human sequences by site directed mutagenesis of individual residues or by grating of entire complementarity determining regions. Alternatively, humanized antibodies may be produced using recombinant methods (see, e.g., GB2188638B). Antibodies which specifically bind to a peptide of this invention may contain antigen binding sites which are either partially or fully humanized, as disclosed in U.S. Pat. No. 5,565,332.

Alternatively, techniques described for the production of single chain antibodies may be adapted using methods known in the art to produce single chain antibodies which specifically bind to a peptide of this invention. Antibodies with related specificity, but of distinct idiotypic composition, can be generated by chain shuffling from random combinatorial immunoglobin libraries (Burton, Proc. Natl. Acad. Sci. 88:11120-23, 1991).

Single-chain antibodies also may be constructed using a DNA amplification method, such as PCR, using hybridoma cDNA as a template (Thirion, et al., Eur. J. Cancer Prev. 5:507-11, 1996). Single-chain antibodies can be mono- or bispecific, and can be bivalent or tetravalent. Construction of tetravalent, bispecific single-chain antibodies is taught, for example, in Coloma & Morrison (Nat. Biotechnol. 15:159-63, 1997). Construction of bivalent, bispecific single-chain antibodies is taught in Mallender & Voss (J. Biol. Chem. 269:199-206, 1994).

A nucleotide sequence encoding a single-chain antibody may be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar, et al., Int. J. Cancer 61:497-501, 1995; Nicholls, et al., J. Immunol. Meth. 165:81-91, 1993).

Antibodies which specifically bind to a peptide of this invention may also be produced by inducing in vivo production in the lymphocyte population or by screening immunoglobulin libraries or panels of highly specific binding reagents as disclosed in the literature (Orlandi, et al., Proc. Natl. Acad. Sci. 86:38333-37, 1989; Winter, et al., Nature 349:293-99, 1991).

Other types of antibodies may be constructed and used therapeutically in methods of the invention. For example, chimeric antibodies may be constructed as disclosed in WO 93/03151. Binding proteins which are derived from immunoglobulins and which are multivalent and multispecific, such as the “diabodies” also can be prepared (see, e.g., WO 94/13804,).

Human antibodies with the ability to bind to the peptides of this invention may also be identified from the MorphoSys HuCAL® library as follows. A peptide of this invention may be coated on a microtiter plate and incubated with the MorphoSys HuCAL® Fab phage library. Those phage-linked Fabs not binding to the peptide of this invention can be washed away from the plate, leaving only phage which tightly bind to the peptide of this invention. The bound phage can be eluted, for example, by a change in pH or by elution with E. coli and amplified by infection of E. coli hosts. This panning process can be repeated once or twice to enrich for a population of antibodies that tightly bind to the peptide of this invention. The Fabs from the enriched pool are then expressed, purified, and screened in an ELISA assay.

Antibodies according to the invention may be purified by methods well known in the art. For example, antibodies may be affinity purified by passage over a column to which a peptide of this invention is bound. The bound antibodies can then be eluted from the column using a buffer with a high salt concentration.

Methods of Use

As used herein, various terms are defined below.

When introducing elements of the present invention or embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy” or “co-therapy” means the administration of two or more therapeutic agents. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in the disease condition, while avoiding or minimizing adverse side effects associated with the given therapeutic treatment.

The term “pharmaceutically acceptable” means that the subject item is appropriate for use in a pharmaceutical product.

The peptides of the present invention may be used for reducing systemic inflammatory response resulting in a multitude of homeostatic changes such as ischemic reperfusion injury and increased blood loss. Theses peptides may also be uses to reduce perioperative blood loss, for example, during cardiovascular surgeries (e.g., coronary artery bypass, off-pump, valve, vascular, lung-volume reduction and Cox-Maze procedures), orthopedic surgeries (e.g., spine, hip replacement and repair, knee replacement and tumor resection), neurosurgery, reconstructive (plastic) surgery, and oncology surgeries.

The peptides of the present invention may also be used in the treatment of trauma (including multi-organ dysfunction and brain injury), ischemia reperfusion injury (e.g., stroke, intracerebral hemorrhage, myocardial Infarction, transplant preservation, and anterior cruciate ligament), cancer (e.g., metastasis and primary tumor suppression), lung ciliary functions (e.g., asthma, cystic fibrosis, chronic obstructive pulmonary disease and antitrypsin deficiency) and organ transplant procedures (e.g., post-cadaveric organ preservation and transplant surgery). The peptides of the present invention may also be used in applications such as fibrin glues (e.g., for use during spinal taps, treating surgical wounds, and dental surgery).

The peptides of the present invention may be used alone or in combination with additional therapies and/or compounds known to those skilled in the art. Alternatively, the methods and peptides described herein may be used, partially or completely, in combination therapy. Such co-therapies may be administered in any combination of two or more drugs. Such co-therapies may be administered in the form of pharmaceutical compositions, as described above.

Based on well known assays used to determine the efficacy for treatment of conditions identified above in mammals, and by comparison of these results with the results of known medicaments that are used to treat these conditions, the effective dosage of the peptides of this invention can readily be determined for treatment of each desired indication. The amount of the active ingredient (e.g., peptides) to be administered in the treatment of one of these conditions can vary widely according to such considerations as the particular peptide and dosage unit employed, the mode of administration, the period of treatment, the age and sex of the patient treated, and the nature and extent of the condition treated.

The total amount of the active ingredient to be administered may generally range from about 0.0001 mg/kg to about 200 mg/kg, or from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific peptide employed, the age of the patient, the diet of the patient, time of administration, route of administration, rate of excretion of the drug, drug combinations, and the like. The desired mode of treatment and number of doses of a peptide of the present invention may be ascertained by those skilled in the art using conventional treatment tests.

The peptides of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which are comprised of a pharmaceutically acceptable carrier and a therapeutically effective amount of a peptide. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a peptide is that amount which produces a result or exerts an influence on the particular condition being treated. The peptides described herein may be administered with a pharmaceutically-acceptable carrier using any effective conventional dosage unit forms, including, for example, immediate and timed release preparations, orally, parenterally, topically, or the like.

For oral administration, the peptides may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

The peptides of this invention may also be administered parenterally, that is, subcutaneously, intravenously, intramuscularly, or interperitoneally, as injectable dosages of the peptide in a physiologically acceptable diluent with a pharmaceutical carrier which may be a sterile liquid or mixture of liquids with or without the addition of a pharmaceutically acceptable surfactant or emulsifying agent or other pharmaceutical adjuvants.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

The pharmaceutical compositions may be in the form of sterile injectable aqueous suspensions. Such suspensions may be formulated according to known methods using suitable dispersing or wetting agents and suspending agents.

A composition of the invention may also be administered in the form of suppositories for rectal administration of the drug. These compositions may be prepared by mixing the drug (e.g., peptide) with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the peptides of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

The peptides described herein may be administered as the sole pharmaceutical agent or in combination with one or more other pharmaceutical agents where the combination causes no unacceptable adverse effects.

The peptides described herein may also be utilized, in compositions, in research and diagnostics, or as analytical reference standards, and the like. Therefore, the present invention includes compositions which are comprised of an inert carrier and an effective amount of a peptide identified by the methods described herein, or a salt or ester thereof. An inert carrier is any material which does not interact with the peptide to be carried and which lends support, means of conveyance, bulk, traceable material, and the like to the peptide to be carried. An effective amount of peptide is that amount which produces a result or exerts an influence on the particular procedure being performed.

Peptides are known to undergo hydrolysis, deamidation, oxidation, racemization and isomerization in aqueous and non-aqueous environment. Degradation such as hydrolysis, deamidation or oxidation can readily detected by capillary electrophoresis. Enzymatic degradation notwithstanding, peptides having a prolonged plasma half-life, or biological resident time, should, at minimum, be stable in aqueous solution. It is essential that peptide exhibits less than 10% degradation over a period of one day at body temperature. It is still more preferable that the peptide exhibits less than 5% degradation over a period of one day at body temperature. Stability (i.e., less than a few percent of degradation) over a period of weeks at body temperature will allow less frequent dosing. Stability in the magnitude of years at refrigeration temperature will allow the manufacturer to present a liquid formulation, thus avoid the inconvenience of reconstitution. Additionally, stability in organic solvent would provide peptide be formulated into novel dosage forms such as implant.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Production and Refolding of Aprotinin

Aprotinin may be produced by expression in E. coli, yeast, insect cells, mammalian cells, or transgenic plants using methods known to those skilled in the art (e.g., Staley, Proc. Natl. Acad. Sci. 89:1519-1523, 1992; Azzoni, Biotechnol. Bioeng. 80:268-276, 2002; Auerswald, Biol. Che,. Hoppe-Seyler 368:1413-1425, 1987) or synthesized using solid-phase peptide synthesis (e.g., Ferrer, Int. J. Pept. Protein Res. 40:194-207, 1992). If expressed in the disulfide-reduced form, aprotinin may be refolded using methods known to those skilled in the art (e.g., Ferrer, 1992; Staley, 1992; Azzoni, 2002).

For expression in E. coli, an expression vector is prepared by ligating a synthetic gene encoding SEQ ID NO: 15 using codons chosen for optimal E. coli usage into pET-3a or any other suitable E. coli expression vector. The plasmid is transformed into E. coli strain BL21 (DE3) pLysS and expression is induced with IPTG. The cells are harvested with centrifugation and lysed with sonication. The insoluble cell lysate fraction is resuspended in 8 M urea and dialyzed against 10% acetic acid. The aprotinin variant is then purified using C₁₈ reversed phase HPLC. The aprotinin variant is refolded in a redox buffer containing reduced and oxidized glutathione and purified with C₁₈ reversed phase HPLC.

Aprotinin variants are also produced using solid-phase peptide synthesis. The peptides are synthesized with an Applied Biosystems 433A peptide synthesizer using Fmoc or Boc chemistry with HBTU activation on Wang Rink amide resin or on any other suitable resin. The peptides are cleaved with 84.6% TFA, 4.4% phenol, 4.4% water, 4.4% thioanisol, and 2.2% ethanedithiol; and the peptides are precipitated from the cleavage cocktail using cold tertbutylmethyl ether. The precipitate is washed with cold ether and dried under argon. The peptides are purified with by reversed phase C₁₈ HPLC with linear water/acetonitrile gradients containing 0.1% TFA. The aprotinin variants are then refolded using methods known to those skilled in the art (e.g., Ferrer, Int. J. Pept. Protein Res. 40:194-207, 1992; Staley, 1992; Azzoni, 2002).

### Example 2

### PEGylation of Aprotinin Variants

PEG derivatives are prepared by incubating methoxypolyethlene glycols derivitized with maledimide for coupling to the mercapto moiety of the N-terminal modifying group. mPEG-MAL or mPEG2-MAL products supplied by Nektar Therapeutics (Huntsville, Ala., USA) or GLE-200MA or GLE-400MA products supplied by NOF (Toyko, Japan) may be used. Coupling reactions are performed by incubating aprotinin and a two-fold molar excess of maleimide-PEG in 50 mM Tris, pH 7 at room temperature for 2-12 hours. The preferred aprotinin concentration is 1 mg/ml or less. Underivatized aprotinin variants and PEG are purified from the PEGylated aprotinin variant with ion exchange chromatography and dialysis or by reversed phase C₁₈ HPLC.

### Example 3

### Determination of in vitro Protease Inhibition Activity

Inhibition of proteases such as trypsin, plasma kallikrein, and plasmin by the aprotinin variants disclosed here may be assayed using spectroscopic assays known to those skilled in the art.

For kallikrein inhibition, 1 unit of protease is diluted in 16 ml 50 mM Tris, 0.1 M NaCl, and 0.05% Tween 20, pH 8.2. This enzyme solution (200 μl) is mixed with decreasing volumes of test buffer (e.g., 250, 240, 230, 220, 200, 180, 170, 150, 100, and 50 μl) and increasing amounts of inhibitor (e.g., 10, 20, 30, 50, 70, 80, 100, 150, 200, and 250 μl at 0.7 mg/ml) are added. The kallikrein/inhibitor solution are incubated at room temperature for 4 hours. An aliquot (180 μl) of each solution is added to 20 μl of substrate solution, and the reaction monitored by the change in absorption. Suitable substrates include: S-2302 for kallekrein; chromozym PL for plasmin; HD-Pro-Phe-Arg-pNA for factor Xi, S-2444 for trypsin, and Suc-Phe-Leu-Phe-pNA for chrymotrypsin.

### Example 4

### Determination of Pharmacokinetic Properties of Aprotinin Variants

The plasma levels of the aprotinin variants of the present invention in animal models such as mice, rats, dogs, and monkeys may be determined following iv infusion of the aprotinin variant. Aprotinin variant levels are measured using a sandwich ELISA that utilizes a capture antibody to aprotinin (produced as described in Example 6) and a reporter antibody to PEG (e.g., AGP3 from Acadmica Sinica). Aprotinin variant plasma levels may also be measured using radiolabeled aprotinin variants (e.g., Shin, Pharm. Pharmcol. Commun. 4:257-260, 1998).

### Example 5

### Determination of the In Vivo Effects of Aprotinin Variants in Animals

The effects of aprotinin variants on blood loss are determined following transection of the tails of anesthetized rats. The rats are treated with Plavix (3 mg/kg). Two hours later the rats are anesthetized with pentobarbital (80 mg/kg, i.p.) and treated with aprotinin (10 mg/kg, i.v.). Ten minutes later, the distal 2 mm of tail is removed and placed in to saline. The time for bleeding to stop is measured. Aprotinin and active variants reduce the bleeding time of the Plavix-treated group.

### Example 6

### Production of Aprotinin Antibodies

Synthesis of peptides derived from the aprotinin sequence with an additional N or C-terminal Cys residue are performed as described in Example 1. Peptide identity is confirmed with MALDI mass spectrometry using a PerSeptive V Biosystems Voyager DE Pro MALDI mass spectrometer. The cysteine residue is coupled to KLH using the Pierce Imject Maleimide Activated mcKLH kit and protocol (Pierce, Rockford, Ill.). Rabbits are immunized and antibodies isolated using procedures known to those of skilled in the art. The antibodies produced in rabbits to the aprotinin peptide are confirmed by an enzyme-linked immunoadsorbent assay (ELISA) using methods known to those of skilled in the art.

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

TABLE 1

| | |
|---|---|
| SEQ ID | |
| NO | SEQUENCE |

| | |
|---|---|
| 1 | RPDFCLEPPY TGPCKARIIR YFYNAKAGLC |
| (aprotinin) | QTFVYGGCRA KRNNFKSAED CMRTCGGA |
| 2 | RDFCLEPPST GPCRAAIIRY FYDATAGLCE |
| | TFVYGGCRAN RNNFKSAEDC METCGGA |
| 3 | MAQLCGLRRS RAFLALLGSL LLSGVLAADR |
| bikunin | ERSIHDFCLV SKVVGRCRAS MPRWWYNVTD |
| | GSCQLFVYGG CDGNSNNYLT KEECLKKCAT |
| | VTENATGDLA TSRNAADSSV PSAPRRQDSE |
| | DHSSDMFNYE EYCTANAVTG PCRASFPRWY |
| | FDVERNSCNN FIYGGCRGNK NSYRSEEACM |
| | LRCFRQQENP PLPLGSKVVV LAGLFVMVLI |
| | LFLGASMVYL IRVARRNQER ALRTVWSSGD |
| | DKEQLVKNTY VL |
| 4 | RPDFCLEPPY TGPAKARIIR YFYNAKAGLA |
| | QTFVYGGARA KRNNFKSAED AMRTCGGA |
| 5 | RPDFCLEPPY TGPCKARIIR YFYNAKAGLA |
| | QTFVYGGCRA KRNNFKSAED AMRTCGGA |
| 6 | RPDFALEPPY TGPCKARIIR YFYNAKAGLC |
| | QTFVYGGCRA KRNNFKSAED CMRTAGGA |
| 7 | TPG CDTSNQAKAQ RPDFCLEPPY TGPCKARIIR |
| | YFYNAKAGLC QTFVYGGCRA KRNNFKSAED |
| | CMRTCGGA |
| 8 | CDTSNQAKAQ RPDFCLEPPY TGPCKARIIR |
| | YFYNAKAGLC QTFVYGGCRA KRNNFKSAED |
| | CMRTCGGA |
| 9 | RPDFCLEPPY TGPCKARIIR YFYNAKAGLC |
| | QTFVYGGCRA KRNNFKSAED CMRTCGGA |
| | SGGSGGSGGCSGG |
| 10 | RPDFCLEPPY TGPCKARIIR YFYNAKAGLC |
| | QTFVYGGCRA KRNNFKSAED CMRTCGGA |
| | SGGSGGSGGC |
| 11 | TPG CDTSNQAKAQ RDFCLEPPST GPCRAAIIRY |
| | FYDATAGLCE TFVYGGCRAN RNNFKSAEDC |
| | METCGGA |
| 12 | CDTSNQAKAQ RDFCLEPPST GPCRAAIIRY |
| | FYDATAGLCE TFVYGGCRAN RNNFKSAEDC |
| | METCGGA |
| 13 | RDFCLEPPST GPCRAAIIRY FYDATAGLCE |
| | TFVYGGCRAN RNNFKSAEDC METCGGA |
| | SGGSGGSGGC SGG |
| 14 | RDFCLEPPST GPCRAAIIRY FYDATAGLCE |
| | TFVYGGCRAN RNNFKSAEDC METCGGA |
| | SGGSGGSGGC |
| 15 | A₁A₂A₃A₄A₅A₆A₇A₈A₉A₁₀ RPDFCLEPPY |
| | TGPCKARIIR YFYNAKAGLC QTFVYGGCRA |
| | KRNNFKSAED CMRTCGG A₁₁A₁₂A₁₃A₁₄A₁₅A₁₆ |
| | A₁₇A₁₈A₁₉A₂₀ |

TABLE 2

| PEG reagent | Structure |
|---|---|
| Linear PEGmPEG-MAL(e.g., Nektar 2D2M0H01and 2D2M0P01) | 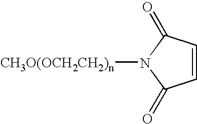 |
| Branched PEGmPEG2-MAL(e.g., Nektar 2D3X0T01) | 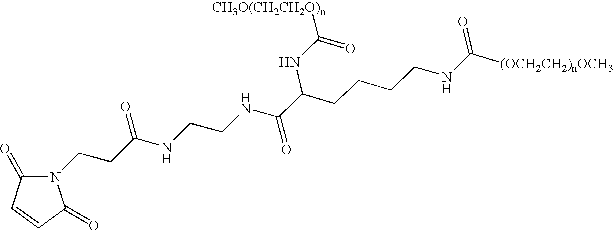 |
| Branched PEG(e.g., NOF GL2-400MA) | 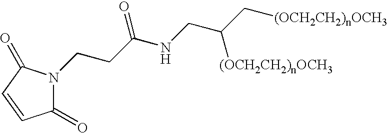 |

We claim:

1. A peptide selected from the group consisting of SEQ ID NO: 4, 5, 6, 7, 8, 9, 10, 11, 12, 13, 14, 15, and functionally equivalent fragments, derivatives, and variants thereof.

2. The peptide of claim 1, wherein the peptide is PEGylated.

3. A pharmaceutical composition comprising a therapeutically effective amount of a peptide of claim 1 or 2, in combination with a pharmaceutically acceptable carrier.

4. A pharmaceutical composition comprising a therapeutically effective amount of a peptide of claim 1 or 2, in combination with a pharmaceutically acceptable carrier and one or more pharmaceutical agents.

5. A method for reducing perioperative blood loss comprising the step of administering to a subject in need thereof a therapeutically effective amount of a peptide of claim 1 or claim 2.

6. The method of claim 5, wherein the peptide is administered to reduce perioperative blood loss during cardiovascular surgery, orthopedic surgery, neurosurgery, reconstructive surgery, or oncology surgery.

7. A method for reducing systemic inflammatory response comprising the step of administering to a subject in need thereof a therapeutically effective amount of a peptide of claim 1 or claim 2.

8. A method for the treatment of ischemia reperfusion injury comprising the step of administering to a subject in need thereof a therapeutically effective amount of a peptide of claim 1 or claim 2.

9. A method for the treatment of cancer comprising the step of administering to a subject in need thereof a therapeutically effective amount of a peptide of claim 1 or claim 2.

10. A method for the treatment of stroke or intracerebral hemorrhage comprising the step of administering to a subject in need thereof a therapeutically effective amount of a peptide of claim 1 or claim 2.

11. A method for the treatment of myocardial infarction comprising the step of administering to a subject in need thereof a therapeutically effective amount of a peptide of claim 1 or claim 2.

12. A method for the treatment of asthma, cystic fibrosis, and chronic obstructive pulmonary disease comprising the step of administering to a subject in need thereof a therapeutically effective amount of a peptide of claim 1 or claim 2.

13. A fibrin glue comprising a peptide of claim 1 and a pharmaceutically acceptable carrier.

14. A polynucleotide encoding a peptide of claim 1, or a degenerate variant thereof.

15. A vector comprising a polynucleotide of claim 14.

16. A host cell comprising a vector of claim 15.

17. A method for producing a peptide comprising:
a) culturing the host cell of claim 16 under conditions suitable for the expression of said polypeptide; and
b) recovering the polypeptide from the host cell culture.

18. A purified antibody which binds specifically to the polypeptide of claim 1.

19. Peptides according to claim 1 for reducing perioperative blood loss.

20. Medicament containing at least one peptide according to claim 1 in combination with at least one pharmaceutically acceptable, pharmaceutically safe carrier or excipient.

21. Use of peptides according to claim 1 for manufacturing a medicament for reducing perioperative blood loss.

22. Medicaments according to claim 20 for reducing perioperative blood loss.


SEQUENCE LISTING (24 sequences)

SEQ ID NO: 1 - protein, 58 aa, Bos taurus [sequence omitted]

SEQ ID NO: 2 - protein, 57 aa, Bos taurus
RDFCLEPPSTGPCRAAIIRYFYDATAGLCETFVYGGCRANRNNFKSAEDCMETCGGA

SEQ ID NO: 3 - protein, 252 aa, Homo sapiens
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 4 - protein, 58 aa, Bos taurus
RPDFCLEPPYTGPAKARIIRYFYNAKAGLAQTFVYGGARAKRNNFKSAEDAMRTCGGA

SEQ ID NO: 5 - protein, 58 aa, Bos taurus
RPDFCLEPPYTGPCKARIIRYFYNAKAGLAQTFVYGGCRAKRNNFKSAEDAMRTCGGA

SEQ ID NO: 6 - protein, 58 aa, Bos taurus
RPDFALEPPYTGPCKARIIRYFYNAKAGLCQTFVYGGCRAKRNNFKSAEDCMRTAGGA

SEQ ID NO: 7 - protein, 71 aa, Bos taurus
TPGCDTSNQAKAQRPDFCLEPPYTGPCKARIIRYFYNAKAGLCQTFVYGGCRAKRNNFKSAEDCMRTCGGA

SEQ ID NO: 8 - protein, 68 aa, Bos taurus
CDTSNQAKAQRPDFCLEPPYTGPCKARIIRYFYNAKAGLCQTFVYGGCRAKRNNFKSAEDCMRTCGGA

SEQ ID NO: 9 - protein, 71 aa, Bos taurus
RPDFCLEPPYTGPCKARIIRYFYNAKAGLCQTFVYGGCRAKRNNFKSAEDCMRTCGGASGGSGGSGGCSGG

SEQ ID NO: 10 - protein, 68 aa, Bos taurus
RPDFCLEPPYTGPCKARIIRYFYNAKAGLCQTFVYGGCRAKRNNFKSAEDCMRTCGGASGGSGGSGGC

SEQ ID NO: 11 - protein, 70 aa, Bos taurus
TPGCDTSNQAKAQRDFCLEPPSTGPCRAAIIRYFYDATAGLCETFVYGGCRANRNNFKSAEDCMETCGGA

SEQ ID NO: 12 - protein, 67 aa, Bos taurus
CDTSNQAKAQRDFCLEPPSTGPCRAAIIRYFYDATAGLCETFVYGGCRANRNNFKSAEDCMETCGGA

SEQ ID NO: 13 - protein, 70 aa, Bos taurus
RDFCLEPPSTGPCRAAIIRYFYDATAGLCETFVYGGCRANRNNFKSAEDCMETCGGASGGSGGSGGCSGG

SEQ ID NO: 14 - protein, 67 aa, Bos taurus
RDFCLEPPSTGPCRAAIIRYFYDATAGLCETFVYGGCRANRNNFKSAEDCMETCGGASGGSGGSGGC

SEQ ID NO: 15 - protein, 77 aa, Bos taurus
  MISC_FEATURE (1)..(77): Xaa can be any naturally occurring amino acid, any unnatural amino acid, or deleted.
XXXXXXXXXXRPDFCLEPPYTGPCKARIIRYFYNAKAGLCQTFVYGGCRAKRNNFKSAEDCMRTCGGXXXXXXXXXX

SEQ ID NO: 16 - protein, 58 aa, Homo sapiens
IHDFCLVSKVVGRCRASMPRWWYNVTDGSCQLFVYGGCDGNSNNYLTKEECLKKCATV

SEQ ID NO: 17 - protein, 58 aa, Homo sapiens
YEEYCTANAVTGPCRASFPRWYFDVERNSCNNFIYGGCRGNKNSYRSEEACMLRCFRQ

SEQ ID NO: 18 - protein, 58 aa, Homo sapiens
DKGHCVDLPDTGLCKESIPRWYYNPFSEHCARFTYGGCYGNKNNFEEEQQCLESCRGI

SEQ ID NO: 19 - protein, 58 aa, Homo sapiens; identical to SEQ ID NO: 16

SEQ ID NO: 20 - protein, 58 aa, Homo sapiens
LPNVCAFPMEKGPCQTYMTRWFFNFETGECELFAYGGCGGNSNNFLRKEKCEKFCKFT

SEQ ID NO: 21 - protein, 58 aa, Homo sapiens
KEDSCQLGYSAGPCMGMTSRYFYNGTSMACETFQYGGCMGNGNNFVTEKECLQTCRTV

SEQ ID NO: 22 - protein, 58 aa, Homo sapiens
ETDICKLPKDEGTCRDFILKWYYDPNTKSCARFWYGGCGGNENKFGSQKECEKVCAPV

SEQ ID NO: 23 - protein, 58 aa, Homo sapiens
KPDFCFLEEDPGICRGYITRYFYNNQTKQCERFKYGGCLGNMNNFETLEECKNICEDG

SEQ ID NO: 24 - protein, 60 aa, Homo sapiens
SDDPCSLPLDEGSCTAYTLRWYHRAVTGSTEACHPFVYGGCGGNANRFGTREACERRCPP